(19) United States
(12) Patent Application Publication
(10) Pub. No.: US 2024/0376043 A1
(43) Pub. Date: Nov. 14, 2024

(54) CHEMICAL PRODUCTION ASSEMBLY FOR ISOCYANATES

(71) Applicant: BASF SE, Ludwigshafen am Rhein (DE)

(72) Inventors: Maximilian KOELLER, Schwarzheide (DE); Johannes BUETTNER, Schwarzheide (DE); Ruediger FRITZ, Bernsdorf (DE); Jonas MATTERN, Schwarzheide (DE); Jan Pablo JOSCH, Ludwigshafen am Rhein (DE)

(21) Appl. No.: 18/692,963

(22) PCT Filed: Sep. 19, 2022
(86) PCT No.: PCT/EP2022/075966

§ 371 Date: Mar. 18, 2024

(30) Foreign Application Priority Data
Sep. 20, 2021 (EP) 21197811.9

(51) Int. Cl. C07C 263/10; B01J 19/00

(52) U.S. Cl. CPC C07C 263/10; B01J 19/0006; B01J 2219/0004

(57) ABSTRACT

A chemical production assembly for producing an isocyanate. comprising n serially arranged units U (i), i=1 . . . n, n≥2. wherein a unit U(i) is for preparing a chemical product cp(i) at a preparation rate PR(i) by using, as starting material. a chemical product cp(i+1) preprared in the unit U(i+1) ar—ranged upstream of said unit U(i), wherein said unit U(i) comprises an inlet means for receiving said chemical product cp(i+1) at an input rate IR(i). said unit U(i) being characterized by a nominal preparation rate PRN(i) and a nominal input rate IRN(i); and a unit U(i+1), i=1 . . . n-1, is for preparing the chemical product cp(i+1) and for supplying said chemical product cp(i+1) to the inlet means of the unit U(i) arranged downstream of said unit U(i+1) at a supply rate SR(i+1) with SR(i+1)=IR(i).

The present invention relates to a chemical production assembly for producing an isocyanate, to a process for producing isocyanate being carried out in such a chemical production assembly and to the use of such a production assembly and/or such a process.

A chemical production assembly for producing an isocyanate typically comprises more than one unit and at least some units are serially arranged. In the terms of this application, such a chemical production assembly comprises n serially arranged units U(i), i=1 . . . n, n≥2. Each unit U(i) is designed for preparing a chemical product cp(i) at a preparation rate PR(i) by using, as starting material, a chemical product cp(i+1) prepared in the unit U(i+1) arranged upstream of said unit U(i). Each unit U(i) comprises an inlet means for receiving said chemical product cp(i+1) at an input rate IR(i). Further, each unit U(i) is characterized by a nominal preparation rate PR_{N}(i) and a nominal input rate IRN(i). Because of the serial character, for at least one unit u(i), a unit U(i+1) is arranged upstream of the unit u(i) for preparing the chemical product cp(i+1) and for supplying said chemical product cp(i+1) to the inlet means of the unit U(i) arranged downstream of said unit U(i+1) at a supply rate SR(i+1). This supply rate SR(i+1) of the unit U(i+1) is equal to the input rate IR(i) of the unit U(i): SR(i+1)=IR(i).

According to the definitions chosen in this application, the unit U(i+1) comprises a sub-unit SU(i+1) for preparing the chemical product cp(i+1). This sub-unit SU(i+1) comprises an inlet means for receiving a chemical product at an input rate IR(i+1) and an outlet means for removing said chemical product cp(i+1) from SU(i+1). The term “preparing” is to be understood in the broadest sense, meaning that the received chemical product is processed in a chemical and/or physical way. The chemical product cp(i+1) is prepared by the sub-unit SU(i+1) at a preparation rate PR(i+1) and said sub-unit SU(i+1) is characterized by a nominal preparation rate PR_{N}(i+1) and a nominal input rate IR_{N}(i+1).

In practice, each unit of the production assembly needs maintenance, often in a regular time pattern, such that it has a production mode and a maintenance mode. During maintenance, it is usually necessary to shut down the unit such that the chain of the production line is interrupted. If all units of the chemical production assembly had the same maintenance pattern, the whole chemical production unit could be shut down for maintenance and started-up after the whole chemical production assembly has been maintained with a minimum loss of production time, but the maintenance time patterns of the different units are usually different from one another.

So, the problem arises that the maintenance times of the units add-up, leading to a loss of production time.

It is therefore an object of the invention to provide an improved chemical production assembly that allows a more efficient production.

According to the invention, at least one unit U(i+1) comprises a dynamic storage means DS(i+1) for temporary storage of chemical product cp(i+1) prepared by the sub-unit SU(i+1), wherein said dynamic storage means DS(i+1) comprises an inlet means being connected to the outlet means of the sub-unit SU(i+1) for receiving chemical product cp(i+1) from SU(i+1), and further comprises an outlet means for removing chemical product cp(i+1) from DS(i+1) at the supply rate SR(i+1). This outlet means is connected to the inlet means of the unit U(i) arranged downstream of U(i+1). The dynamic storage means DS(i+1) has a storage capacity SC(i+1) and is characterized by a dynamic storage rate DR(i+1) with DR(i+1)=PR(i+1)-SR(i+1). This dynamic storage rate is different from zero (DR(i+1)≠0) if at least one of the unit u(i) and the sub-unit SU(i+1) works at or near its nominal rate, such that at least one of the ratios IR (i): IR_{N} (i) and IR(i+1): IR_{N} (i+1) is in the range of from 0.95:1 to 1.05:1. In other words: The dynamic storage means empties or fills when at least one of the unit U(i) and the sub-unit SU(i+1) works at its nominal rate, such that it constitutes a buffer that can be used during maintenance of the unit U(i) and the sub-unit SU(i+1).

As has been mentioned, usually each unit U(i) exhibits a working mode and a maintenance mode. The working mode is characterized by IR(i)=IR_{N}(i) and PR(i)=PR_{N}(i) and the maintenance mode is characterized by IR(i)=PR(i)=0.

As has also already been mentioned, the at least one unit U(i) usually further exhibits a start-up mode. This mode is the mode “between” the maintenance mode and the working mode and is characterized by 0<IR(i)<IR_{N}(i) and 0<PR(i)<PR_{N}(i).

As discussed above, the at least one unit U(i) often exhibits a regular maintenance pattern with a regular maintenance time Δt_{MM}(i) for which the unit U(i) is in its maintenance mode during which IR(i)=0. It is desired that SU(i+1) can continue to work at its nominal preparation rate PR_{N}(i+1) when the unit U(i) is in its maintenance mode and thus it is preferred that the dynamic storage capacity SC(i+1) is large enough to receive all of the chemical product produced by the sub-unit SU(i+1) during a regular maintenance time of the unit U(i): SC(i+1)>Δt_{MM}(i)×PR_{N}(i+1).

Thus, the nominal input rate IR_{N}(i) is the “target status/desired status” (known in the German language as Soll-Zustand) of the unit with respect to the input rate during its working mode. Similarly, the nominal production rate PR_{N}(i) is the “target status/desired status” of the unit with respect to the production rate during its working mode.

In the same way, the at least one sub-unit SU(i+1) usually exhibits a regular maintenance pattern with a regular maintenance time Δt_{MM}(i+1) for which the sub-unit SU(i+1) is in its maintenance mode. In order to make sure that the unit U(i) can continue to work in its working mode with the nominal input rate IR_{N}(i), the dynamic storage capacity is preferably large enough to deliver all of the chemical product needed by the unit U(i) during a regular maintenance time At_{MM}(i+1) of the sub-unit SU(i+1): SC(i+1)>Δt_{MM}(i+1)×IRN(i).

If—as it is assumed as the normal case—the at least one unit U(i) and the at least one sub-unit SU(i+1) both have a regular maintenance pattern, they both do not only each exhibit a maintenance time Δt_{MM}(i), Δt_{MM}(i+1), they also each exhibit a maintenance interval time Δt_{MI}(i), Δt_{MI}(i+1) between two consecutive maintenance times Δt_{MM}(i), Δt_{MM}(i+1) for which the at least one unit U(i)—respectively the sub-unit SU(i+1)—is not in its maintenance mode. As has been described above, the nominal preparation rate PR_{N}(i+1) and the nominal input rate IRN(i) differ from each other (PR_{N}(i+1)≠IR_{N}(i)) and thus the dynamic storage means is filled or emptied (DR(i+1)≠0) not only when one of the sub-unit SU(i+1) and the unit U(i) is in its maintenance mode (DR(i+1)≠0), but also when the sub-unit U(i+1) and the unit U(i) are both in their working mode. So, the dynamic storage capacity should be large enough to “bridge” the state in which the sub-unit U(i+1) and the unit U(i) are in their working mode. For this reason, it is preferred that the storage capacity is larger than the shorter of the maintenance time intervals multiplied with the absolute value of the dynamic storage range DR(i+1) during this state, which is referred to as the nominal dynamic storage range DR_{N}(i+1): SC(i+1)>min[Δt_{MI}(i),Δt_{MI}(i+1)]×|DR_{N}(i+1)|.

In the case that both, the unit and the sub-unit both have a maintenance pattern comprising a working mode and a maintenance mode, one can calculate an average input rate IR_{A}(i) and an average preparation rate PR_{A}(i) for the unit U(i). The average input rate IR_{A}(i) is the total input during a whole cycle divided by the cycle time and the average preparation rate PR_{A}(i) is the total amount of prepared chemical product cp(i) during a whole cycle divided by the cycle time: PR_{A}(i)=Δt(i)×PR_{N}(i)/(Δt(i)+Mt(i)) with Δt(i) being the time between two maintenance modes. Because of the time in which the unit is in its maintenance mode, the following applies: IR_{A}(i)<IR_{N}(i) and PRA(i)<PR_{N}(i). In the same way, the average input rate IR_{A}(i+1) of the sub-unit SU(i+1) is the total input during a whole cycle divided by the cycle time and the average preparation rate PR_{A}(i+1) is the total amount of prepared chemical product cp(i+1) during a whole cycle divided by the cycle time: Like above, the average rates are less than the nominal rates: IR_{A}(i+1)<IR_{N}(i+1) and PR_{A}(i+1)<PR_{N}(i+1). If the sub-unit SU(i+1) prepares chemical product cp(i+1) exclusively for the unit U(i) (and this is preferred and usually the case), the average rates are equal: IR_{A}(i)=PR_{A}(i+1).

In the above, the start-up time has been neglected, but can of course also be taken into account.

It is preferred that the sub-unit SU(i+1) according to (c. 1) consists of one sub-unit SSU(i+1).

Alternatively, it is preferred that the sub-unit SU(i+1) according to (c. 1) comprises, more preferably consists of, z sub-units SSU(i+1) that are arranged in parallel, with z>1. More preferably, z is in the range of from 2 to 5, more preferably in the range of from 2 to 4.

Preferably at least one of the z sub-units SSU(i+1) operates during the maintenance mode and at least one of the z sub-units SSU(i+1) does not operate during said maintenance mode.

Preferably the at least one sub-unit SU(i+1) according to (c. 1) is one sub-unit SU(i+1).

Preferably at least one unit U(i+1) according to (c) is one unit U(i+1).

In a preferred application of the invention, the chemical production assembly is a production assembly for producing a diisocyanate, preferably for producing a toluene diisocyanate, more preferably for producing one or more of 2,4-toluene diisocyanate (TDI) and 2,6-toluene diisocyanate.

Especially in the just defined case, the chemical production assembly comprises a unit U(i) for preparing, as the chemical product cp(i), an aqueous solution comprising sulfuric acid with the concentration of the sulfuric acid being C_{H2SO4}(i), by using, as the starting material cp(i+1), an aqueous solution comprising sulfuric acid with the concentration of the sulfuric acid being C_{H2SO4}(i+1), wherein C_{H2SO4}(i+1)<c_{HsSO4}(i).

In this case it is further preferred that the sub-unit SU(i+1) of the unit U(i+1) (being arranged upstream of the unit U(i)) is a sub-unit for nitrating an organic compound, preferably toluene, with an aqueous solution comprising nitric acid in the presence of sulfuric acid as a catalyst, wherein preferably nitrotoluene, more preferably dinitrotoluene is obtained, such that an aqueous solution comprising sulfuric acid is obtained as a chemical product cp(i+1). As stated above, the concentration of the sulfuric acid is CH2SO4(i+1). In this case, the dynamic storage means DS(i+1) of the unit U(i+1) is a dynamic storage tank for storing the aqueous solution comprising sulfuric acid which is obtained as a chemical product cp(i+1) in the sub-unit SU(i+1).

For the above defined process, preferably, C_{H2SO4}(i) is in the range from 65 to 96 weight %, more preferably in the range of from 80 to 96 weight %, more preferably in the range of from 86 to 96weight %, based on the weight of the aqueous solution comprising sulfuric acid, and, preferably, C_{H2SO4}(i+1) is in the range from 45 to 85 weight %, more preferably in the range of from 55 to 80weight %, more preferably in the range of from 55 to 80 weight %, based on the weight of the aqueous solution comprising sulfuric acid.

It is preferred that PR_{N}(i), more preferably for obtaining a nitrotoluene, more preferably a dinitrotoluene, is in the range of from 0.5 to 3.5 t/t, more preferably in the range of from 0.75 to 1.75 t/t, more preferably in the range of from 1 to 1.5 t/t. Preferably, PR_{N}(i+1), more preferably for obtaining a nitrotoluene, more preferably a dinitrotoluene, is in the range of from 0.75 to 3.5 t/t, more preferably in the range of from 1 to 2 t/t, more preferably in the range of from 1.25 to 1.8 t/t. Preferably, IR_{N}(i), more preferably for obtaining a nitrotoluene, more preferably a dinitrotoluene, is in the range of from 0.45 to 3.5 t/t, more preferably in the range of from 0.65 to 1.85 t/t, more preferably in the range of from 0.8 to 1.75 t/t. Preferably, IR_{N}(i+1), more preferably for obtaining a nitrotoluene, more preferably a dinitrotoluene, is in the range of from 0.6 to 3.5 t/t, more preferably in the range of from 0.8 to 2.2 t/t, more preferably in the range of from 0.9 to 2 t/t.

Especially in the just described application, Δt_{MM}(i) is preferably in the range of from 0.5 h to 30 d, more preferably in the range of from 1 h to 14 d, more preferably in the range of from 5 h to 7 d. Δt_{MM}(i+1), is preferably in the range of from 0.5 h to 30 d, more preferably in the range of from 1 h to 14 d, more preferably in the range of from 5 h to 7 d. Preferably, Δt_{MI}(i) is in the range of from 1month to 10 years, more preferably in the range of from 3 month to 7 years, more preferably in the range of from 6 month to 5 years. Preferably, Δt_{MI}(i+1) is in the range of from 1 month to 10years, more preferably in the range of from 3 month to 7 years, more preferably in the range of from 6 month to 5 years.

Also especially in the just defined case according to which the chemical production assembly is a production assembly for producing a diisocyanate, preferably for producing a toluene diisocyanate, more preferably for producing one or more of 2,4-toluene diisocyanate (TDI) and 2,6-toluene diisocyanate, the chemical production assembly comprises a unit U(i) according to (a) for nitrating an organic compound, preferably toluene, with an aqueous solution comprising nitric acid in the presence of sulfuric acid as a catalyst, preferably to obtain nitrotoluene, more preferably dinitrotoluene, wherein an aqueous solution comprising sulfuric acid is obtained as a chemical product cp(i), wherein in said obtained aqueous solution, the concentration of the sulfuric acid is C_{H2SO4}(i) by using, as the starting material cp(i+1), an aqueous solution comprising sulfuric acid, wherein the concentration of the sulfuric acid is C_{H2SO4}(i+1), with C_{H2SO4}(i+1)>C_{H2SO4}(i).

In the just described case, the sub-unit SU(i+1) of the unit U(i+1) that is arranged upstream of the unit U(i) is preferably a sub-unit for preparing, as the chemical product cp(i+1), an aqueous solution comprising sulfuric acid, wherein the concentration of the sulfuric acid is C_{H2SO4}(i+1). Consequently, the dynamic storage means DS(i+1) of the unit U(i+1) is a dynamic storage tank for storing the aqueous solution comprising sulfuric acid which is obtained as a chemical product cp(i+1) in the sub-unit SU(i+1).

For the above defined process, C_{H2SO4}(i) is preferably in the range from 65 to 96 weight %, more preferably in the range of from 80 to 96 weight %, more preferably in the range of from 86 to 96weight %, based on the weight of the aqueous solution comprising sulfuric acid. C_{H2SO4}(i+1) is preferably in the range from 45 to 85 weight %, more preferably in the range of from 55 to 80weight %, more preferably in the range of from 55 to 80 weight %, based on the weight of the aqueous solution comprising sulfuric acid.

It is preferred that PR_{N}(i), more preferably for obtaining a nitrotoluene, more preferably a dinitrotoluene, is in the range of from 0.5 to 3.5 t/t, more preferably in the range of from 0.75 to 1.75 t/t, more preferably in the range of from 1 to 1.5 t/t. Preferably, PR_{N}(i+1), more preferably for obtaining a nitrotoluene, more preferably a dinitrotoluene, is in the range of from 0.75 to 3.5 t/t, more preferably in the range of from 1 to 2 t/t, more preferably in the range of from 1.25 to 1.8 t/t. Preferably, IR_{N}(i), more preferably for obtaining a nitrotoluene, more preferably a dinitrotoluene, is in the range of from 0.45 to 3.5 t/t, more preferably in the range of from 0.65 to 1.85 t/t, more preferably in the range of from 0.8 to 1.75 t/t. Preferably, IR_{N}(i+1), more preferably for obtaining a nitrotoluene, more preferably a dinitrotoluene, is in the range of from 0.6 to 3.5 t/t, more preferably in the range of from 0.8 to 2.2 t/t, more preferably in the range of from 0.9 to 2 t/t.

Especially in the just described application, Δt_{MM}(i) is preferably in the range of from 0.5 h to 30 d, more preferably in the range of from 1 h to 14 d, more preferably in the range of from 5 h to 7 d.

Preferably, Δt_{MM}(i+1), is in the range of from 0.5 h to 30 d, more preferably in the range of from 1 h to 14 d, more preferably in the range of from 5h to 7d. Preferably, Δt_{MI}(i) is in the range of from 1 month to 10 years, more preferably in the range of from 3 month to 7 years, more preferably in the range of from 6 month to 5 years. Preferably, Δt_{MI}(i+1) is in the range of from 1 month to 10 years, more preferably in the range of from 3 month to 7 years, more preferably in the range of from 6 month to 5 years.

It is also possible to arrange the units U(i) and U(i+1) in a circle, meaning that both units comprise a sub-unit SU(i), SU(i+1) and a dynamic storage means DS(i), DS(i+1), wherein the outlet means of the dynamic storage means DS(i) is connected to the inlet means of the unit(i+1), namely to the inlet means of its sub-unit SU(i+1) and wherein the outlet means of the dynamic storage means DS(i+1) is connected to the inlet means of the unit(i), namely to the inlet means of its subunit SU(i).

Such a circle-configuration is especially useful in a chemical production assembly for producing a diisocyanate, preferably for producing a toluene diisocyanate, more preferably for producing one or more of 2,4-toluene diisocyanate (TDI) and 2,6-toluene diisocyanate, and especially for defining a circle for an aqueous solution comprising sulfuric acid as a catalyst being present in a nitration step.

Preferably, the present invention relates to a chemical production assembly for producing an isocyanate, comprising 2 serially arranged units U(1) and U(2), wherein
(a) unit U(1) is for preparing a chemical product cp(1) at a preparation rate PR (1) by using, as starting material, a chemical product cp(2) prepared in the unit U(2) arranged upstream of said unit U(1), wherein said unit U(1) comprises an inlet means for receiving said chemical product cp(2) at an input rate IR (1), said unit U(1) being characterized by a nominal preparation rate PR_{N}(1) and a nominal input rate IRN (1);
(b) unit U(2) is for preparing the chemical product cp(2) and for supplying said chemical product cp(2) to the inlet means of the unit U(1) arranged downstream of said unit U(2) at a supply rate SR(2) with SR(2)=IR(1);
(c) the unit U(2) comprises
   (c.1) a sub-unit SU(2) for preparing the chemical product cp(2), wherein said sub-unit SU(2) comprises an inlet means for receiving a chemical product at an input rate IR(2) and an outlet means for removing said chemical product cp(2) from SU(2) at a preparation rate PR(2), said sub-unit SU(2) being characterized by a nominal preparation rate PR_{N}(2) with PR_{N}(2)≠IR_{N}(1) and a nominal input rate IRN (2); and
   (c.2) a dynamic storage means DS(2) for temporary storage of chemical product cp(2) prepared according to (c.1), wherein said dynamic storage means DS(2) comprises an inlet means being connected to the outlet means of SU(2) for receiving chemical product cp(2) from SU(2), and further comprises an outlet means for removing chemical product cp(2) from DS(2) at the supply rate SR(2), said outlet means being connected to the inlet means of the unit U(1) arranged downstream of U(2), said dynamic storage means DS(2) having a storage capacity SC(2) and being characterized by a dynamic storage rate DR(2) with DR(2)=PR(2)−SR(2);
wherein for said unit U(2) according to (c), DR(2)≠0 if at least one of the ratios IR(1):IR_{N} (1) and IR(2):IR_{N}(2) is in the range of from 0.95:1 to 1.05:1.

It is preferred that unit U(1) according to (a) be for nitrating an organic compound, more preferably toluene, with an aqueous solution comprising nitric acid in the presence of sulfuric acid as a catalyst, more preferably to obtain nitrotoluene, more preferably dinitrotoluene, wherein an aqueous solution comprising sulfuric acid is obtained as a chemical product cp(1), wherein in said obtained aqueous solution, the concentration of the sulfuric acid is C_{H2SO}(1) by using, as the starting material cp(2), an aqueous solution comprising sulfuric acid, wherein the concentration of the sulfuric acid is C_{H2SO4(}2), with C_{H2SO4}(2) >C_{H2SO4} (1).

It is preferred that the sub-unit SU(2) of the unit U(2) arranged upstream of the unit U(1) is a subunit for preparing, as the chemical product cp(2), an aqueous solution comprising sulfuric acid, wherein the concentration of the sulfuric acid is C_{H2SO4}(2), wherein the dynamic storage means DS(2) of the unit U(2) is a dynamic storage tank for storing the aqueous solution comprising sulfuric acid which is obtained as a chemical product cp(2) in the sub-unit SU(2).

In the above specified application of the invention it can be preferred that the chemical production assembly further comprises a unit for producing an amino toluene, preferably a diamino toluene, arranged downstream of the unit for nitrating toluene, and further comprising a unit for producing a toluene isocyanate, preferably a toluene diisocyanate, arranged downstream of said unit for producing an amino toluene. It is further preferred that the chemical production assembly additionally comprises a unit for producing phosgene, arranged upstream of the unit for producing a toluene isocyanate.

So, one can see that the invention can especially be used for increasing the interruption-free operation time of an isocyanate production process.

Usually, the unit according to the invention will be comprised in a production plant, preferably in a production plant for producing an isocyanate, more preferably for producing a diisocyanate, more preferably for producing a toluene diisocyanate, more preferably for producing one or more of 2,4-toluene diisocyanate (TDI) and 2,6-toluene diisocyanate. If a mixture comprising 2,4-toluene diisocyanate and 2,6-toluene diisocyanate is produced, the molar ratio of 2,4-toluene diisocyanate relative to 2,6-toluene diisocyanate is preferably in the range of from 1:1 to 6:1, more preferably in the range of from 1:1 to 5:1, such as in the range of from 1:1 to 2:1 like 1.5:1, or such as in the range of from 3.5:1 to 4.5:1 like 4:1. Further, it is conceivable that said diisocyanate comprises or is methylene diphenylisocyanate (MDI) and/or hexamethylene diisocyanate (HDI).

The present invention is further illustrated by the following set of embodiments and combinations of embodiments resulting from the dependencies and back-references as indicated. In particular, it is noted that in each instance where a range of embodiments is mentioned, for example in the context of a term such as “The chemical production assembly of any one of embodiments 1 to 4”,every embodiment in this range is meant to be explicitly disclosed for the skilled person, i.e. the wording of this term is to be understood by the skilled person as being synonymous to “The chemical production assembly of any one of embodiments 1, 2, 3 and 4”. Further, it is explicitly noted that the following set of embodiments is not the set of claims determining the extent of protection, but represents a suitably structured part of the description directed to general and preferred aspects of the present invention.

1. A chemical production assembly for producing an isocyanate, comprising n serially ar—ranged units U(i), i=1 . . . n, n≥2, wherein
(a) a unit U(i) is for preparing a chemical product cp(i) at a preparation rate PR(i) by using, as starting material, a chemical product cp(i+1) prepared in the unit U(i+1) arranged upstream of said unit U(i), wherein said unit U(i) comprises an inlet means for receiving said chemical product cp(i+1) at an input rate IR(i), said unit U(i) being characterized by a nominal preparation rate PR_{N}(i) and a nominal input rate IR_{N}(i);
(b) a unit U(i+1), i=1 . . . n-1, is for preparing the chemical product cp(i+1) and for supplying said chemical product cp(i+1) to the inlet means of the unit U(i) arranged downstream of said unit U(i+1) at a supply rate SR(i+1) with SR(i+1)=IR (i);
(c) at least one unit U(i+1) comprises
   (c.1) a sub-unit SU(i+1) for preparing the chemical product cp(i+1), wherein said subunit SU(i+1) comprises an inlet means for receiving a chemical product at an input rate IR(i+1) and an outlet means for removing said chemical product cp(i+1) from SU(i+1) at a preparation rate PR(i+1), said sub-unit SU(i+1) being characterized by a nominal preparation rate PR_{N}(i+1) with PR_{N}(i+1) #IR_{N}(i) and a nominal input rate IR_{N}(i+1); and
   (c.2) a dynamic storage means DS(i+1) for temporary storage of chemical product cp(i+1) prepared according to (c.1), wherein said dynamic storage means DS(i+1) comprises an inlet means being connected to the outlet means of SU(i+1) for receiving chemical product cp(i+1) from SU(i+1), and further comprises an outlet means for removing chemical product cp(i+1) from DS(i+1) at the supply rate SR(i+1), said outlet means being connected to the inlet means of the unit U(i) arranged downstream of U(i+1), said dynamic storage means DS(i+1) having a storage capacity SC(i+1) and being characterized by a dynamic storage rate DR(i+1) with DR(i+1)=PR(i+1)-SR(i+1);
wherein for said at least one unit U(i+1) according to (c), DR(i+1) #0 if at least one of the ratios IR(i):IR_{N}(i) and IR(i+1):IR_{N}(i+1) is in the range of from 0.95:1 to 1.05:1.

2. The chemical production assembly of embodiment 1, wherein each unit U(i) exhibits a maintenance mode which is characterized by IR(i)=PR(i)=0, and a working mode which is characterized by IR(i)=IR_{N}(i) and PR(i)=PR_{N}(i).

3. The chemical production assembly of embodiment 2, wherein at least one unit U(i) further exhibits a start-up mode which is characterized by IR(i)<IR_{N}(i) and PR(i)<PR_{N}(i).

4. The chemical production assembly of embodiment 2 or 3, wherein at least one unit U (i) according to (a) exhibits a regular maintenance pattern with a regular maintenance time Δ t_{MM}(i) for which the unit U(i) is in its maintenance mode.

5. The chemical production assembly of embodiment 4, wherein SC(i+1)>Δt_{MM}(i)×PR_{N}(i+1).

6. The chemical production assembly of any of embodiments 1 to 5, wherein at least one subunit SU(i+1) according to (c.1) exhibits a regular maintenance pattern with a regular maintenance time Δt_{MM}(i+1) for which the sub-unit SU(i+1) is in its maintenance mode.

7. The chemical production assembly of embodiment 6, wherein SC(i+1)>Δt_{MM}(i+1)×IRN(i).

8. The chemical production assembly of embodiment 7 insofar as embodiment 7 depends on embodiment 5, wherein the regular maintenance pattern of the at least one unit U(i) according to (a) exhibits a maintenance interval time Δt_{MI}(i) between two consecutive maintenance times Δt_{MM}(i) for which the at least one unit U(i) is not in its maintenance mode, and the regular maintenance pattern of the at least one sub-unit U(i+1) according to (c. 1) exhibits a maintenance interval time Δt_{MI}(i+1) between two consecutive maintenance times Δt_{MM}(i+1) for which the at least one sub-unit SU(i+1) is not its' in maintenance mode, wherein SC(i+1)>min[Δt_{MI}(i), Δt_{MI}(i+1)]×|DR(i+1) |.

9. The chemical production assembly of any one of embodiments 1 to 8, wherein for at least one unit U(i) according to (a) and at least one unit U(i+1) according to (b),
the at least one unit U(i) exhibits an average input rate IR_{A}(i) and an average preparation rate PR_{A}(i) with IR_{A}(i) <IR_{N}(i) and PR_{A}(i) <PR_{N}(i); and
the at least one sub-unit SU(i+1) according to (c.1) exhibits an average input rate IR_{A}(i+1) and an average preparation rate PR_{A}(i+1) with IR_{A}(i+1) <IR_{N}(i+1) and PR_{A}(i+1) <PR_{N}(i+1);
and IR_{A}(i)=PR_{A}(i+1).

10. The chemical production assembly of any one of embodiments 1 to 9, wherein the sub-unit SU(i+1) according to (c.1) consists of one sub-unit SSU(i+1).

11. The chemical production assembly of any one of embodiments 1 to 10, wherein the subunit SU(i+1) according to (c.1) comprises, preferably consists of, z sub-units SSU(i+1) that are arranged in parallel, with z>1.

12. The chemical production assembly of embodiment 11, wherein z is in the range of 2 to 5,preferably in the range of 2 to 4.

13. The chemical production assembly of embodiment 11 or 12, wherein at least one of the z sub-units SSU(i+1) operates during the maintenance mode and at least one of the z subunits SSU(i+1) does not operate during the maintenance mode.

14. The chemical production assembly of any one of embodiments 1 to 13, wherein the at least one sub-unit SU(i+1) according to (c. 1) is one sub-unit SU(i+1).

15. The chemical production assembly of any one of embodiments 1 to 14, wherein at least one unit U(i+1) according to (c) is one unit U(i+1).

16. The chemical production assembly of any one of embodiments 1 to 15, being a production assembly for producing a diisocyanate, preferably for producing a toluene diisocyanate, more preferably for producing one or more of 2,4-toluene diisocyanate (TDI) and 2,6-toluene diisocyanate.

17. The chemical production assembly of any one of embodiments 1 to 16, preferably of embodiment 16, comprising a unit U(i) according to (a) for preparing, as the chemical product cp(i), an aqueous solution comprising sulfuric acid with the concentration of the sulfuric acid being C_{H2SO4}(i), by using, as the starting material cp(i+1), an aqueous solution comprising sulfuric acid with the concentration of the sulfuric acid being C_{H2SO4}(i+1), wherein C_{2SO4}(i+1)<C_{2SO4}(i).

18. The chemical production assembly of embodiment 11, wherein the sub-unit SU(i+1) of the unit U(i+1) arranged upstream of the unit U(i) is a sub-unit for nitrating an organic compound, preferably toluene, with an aqueous solution comprising nitric acid in the presence of sulfuric acid as a catalyst, preferably to obtain nitrotoluene, more preferably dinitrotoluene, wherein an aqueous solution comprising sulfuric acid is obtained as a chemical product cp(i+1), wherein in said obtained aqueous solution, the concentration of the sulfuric acid is C_{H2SO4(}i+1), wherein the dynamic storage means DS(i+1) of the unit U(i+1) is a dynamic storage tank for storing the aqueous solution comprising sulfuric acid which is obtained as a chemical product cp(i+1) in the sub-unit SU(i+1).

19. The chemical production assembly of embodiment 17 or 18, wherein C_{H2SO4}(i) is in the range from 65 to 96 weight %, preferably in the range of from 80 to 96 weight %, more preferably in the range of from 86 to 96 weight %, based on the weight of the aqueous solution comprising sulfuric acid.

20. The chemical production assembly of any one of embodiments 17 to 19, wherein CH2SO4(i+1) is in the range from 45 to 85 weight %, preferably in the range of from 55 to 80 weight %, preferably in the range of from 55 to 80 weight %, based on the weight of the aqueous solution comprising sulfuric acid.

21. The chemical production assembly of any one of embodiments 17 to 20, wherein PRN(i), preferably for obtaining a nitrotoluene, more preferably a dinitrotoluene, is in the range of from 0.5 to 3.5 t/t, more preferably in the range of from 0.75 to 1.75 t/t, more preferably in the range of from 1 to 1.5 t/t.

22. The chemical production assembly of any one of embodiments 17 to 21, wherein PR_{N}(i+1), preferably for obtaining a nitrotoluene, more preferably a dinitrotoluene, is in the range of from 0.75 to 3.5 t/t, more preferably in the range of from 1 to 2 t/t, more preferably in the range of from 1.25 to 1.8 t/t.

23. The chemical production assembly of any one of embodiments 17 to 22, wherein IR_{N}(i), preferably for obtaining a nitrotoluene, more preferably a dinitrotoluene, is in the range of from 0.45 to 3.5 t/t, more preferably in the range of from 0.65 to 1.85 t/t, more preferably in the range of from 0.8 to 1.75 t/t.

24. The chemical production assembly of any one of embodiments 17 to 23, wherein IR_{N}(i+1), preferably for obtaining a nitrotoluene, more preferably a dinitrotoluene, is in the range of from 0.6 to 3.5 t/t, more preferably in the range of from 0.8 to 2.2 t/t, more preferably in the range of from 0.9 to 2 t/t.

25. The chemical production assembly of any one of embodiments 17 to 24, preferably embodiment 20, wherein Δt_{MM}(i) is in the range of from 0.5 h to 30 d, preferably in the range of from 1 h to 14 d, more preferably in the range of from 5 h to 7 d.

26. The chemical production assembly of any one of embodiments 17 to 25, wherein Δt_{MM}(i+1), is in the range of from 0.5 h to 30 d, preferably in the range of from 1 h to 14 d, more preferably in the range of from 5 h to 7 d.

27. The chemical production assembly of any one of embodiments 17 to 26, wherein Δt_{MI}(i) is in the range of from 1 month to 10 years, preferably in the range of from 3 month to 7 years, more preferably in the range of from 6 month to 5 years.

28. The chemical production assembly of any one of embodiments 17 to 27, wherein Δt_{MI}(i+1) is in the range of from 1 month to 10 years, preferably in the range of from 3 month to 7years, more preferably in the range of from 6 month to 5 years.

29. The chemical production assembly of any one of embodiments 1 to 27, preferably of embodiment 16, comprising a unit U(i) according to (a) for nitrating an organic compound, preferably toluene, with an aqueous solution comprising nitric acid in the presence of sulfuric acid as a catalyst, preferably to obtain nitrotoluene, more preferably dinitrotoluene, wherein an aqueous solution comprising sulfuric acid is obtained as a chemical product cp(i), wherein in said obtained aqueous solution, the concentration of the sulfuric acid is CH2SO(i) by using, as the starting material cp(i+1), an aqueous solution comprising sulfuric acid, wherein the concentration of the sulfuric acid is C_{H2SO4}(i+1), with C_{H2SO4}(i+1)>C_{H2SO4}(i).

30. The chemical production assembly of embodiment 28, wherein the sub-unit SU(i+1) of the unit U(i+1) arranged upstream of the unit U(i) is a sub-unit for preparing, as the chemical product cp(i+1), an aqueous solution comprising sulfuric acid, wherein the concentration of the sulfuric acid is CH2SO4(i+1), wherein the dynamic storage means DS(i+1) of the unit U(i+1) is a dynamic storage tank for storing the aqueous solution comprising sulfuric acid which is obtained as a chemical product cp(i+1) in the sub-unit SU(i+1).

31. The chemical production assembly of embodiment 16, comprising a unit U(i+1) as defined in embodiment 18, a unit U(i) as defined in embodiment 17, a unit U(i+1) as defined in embodiment 30 and a unit U(i) as defined in embodiment 29, wherein the unit U(i+1) as defined in embodiment 30 is the unit U(i) as defined in embodiment 17 and the unit U(i) as defined in embodiment 29 is the unit U(i+1) as defined in embodiment 18.

32. The chemical production assembly of any one of embodiments 1 to 31, with n=2, comprising 2 serially arranged units U(1) and U(2), wherein
(a) unit U(1) is for preparing a chemical product cp(1) at a preparation rate PR(1) by using, as starting material, a chemical product cp(2) prepared in the unit U(2) arranged upstream of said unit U(1), wherein said unit U(1) comprises an inlet means for receiving said chemical product cp(2) at an input rate IR(1), said unit U(1) being characterized by a nominal preparation rate PR_{N}(1) and a nominal input rate IR_{N}(1);
(b) unit U(2), is for preparing the chemical product cp(2) and for supplying said chemical product cp(2) to the inlet means of the unit U(1) arranged downstream of said unit U(2) at a supply rate SR(2) with SR(2)=IR(1);
(c) the unit U(2) comprises
   (c.1) a sub-unit SU(2) for preparing the chemical product cp(2), wherein said sub-unit SU(2) comprises an inlet means for receiving a chemical product at an input rate IR(2) and an outlet means for removing said chemical product cp(2) from SU(2) at a preparation rate PR(2), said sub-unit SU(2) being characterized by a nominal preparation rate PR_{N}(2) with PR_{N}(2)≠IR_{N}(1) and a nominal input rate IR_{N}(2); and
   (c.2) a dynamic storage means DS(2) for temporary storage of chemical product cp(2) prepared according to (c.1), wherein said dynamic storage means DS(2) comprises an inlet means being connected to the outlet means of SU(2) for receiving chemical product cp(2) from SU(2), and further comprises an outlet means for removing chemical product cp(2) from DS(2) at the supply rate SR(2), said outlet means being connected to the inlet means of the unit U(1) arranged downstream of U(2), said dynamic storage means DS(2) having a storage capacity SC(2) and being characterized by a dynamic storage rate DR(2) with DR(2)=PR(2)−SR(2);
wherein for said unit U(2) according to (c), DR(2)≠0 if at least one of the ratios IR(1): IR_{N} (1) and IR(2): IR_{N}(2) is in the range of from 0.95:1 to 1.05:1.

33. The chemical production assembly of embodiment 32, wherein unit U(1) according to (a) is for nitrating an organic compound, preferably toluene, with an aqueous solution comprising nitric acid in the presence of sulfuric acid as a catalyst, preferably to obtain nitrotoluene, more preferably dinitrotoluene, wherein an aqueous solution comprising sulfuric acid is obtained as a chemical product cp(1), wherein in said obtained aqueous solution, the concentration of the sulfuric acid is C_{H2SO}(1) by using, as the starting material cp(2), an aqueous solution comprising sulfuric acid, wherein the concentration of the sulfuric acid is C_{H2SO4}(2), with C_{H2SO4}(2)>C_{H2SO4}(1).

34. The chemical production assembly of embodiment 33, wherein the sub-unit SU(2) of the unit U(2) arranged upstream of the unit U(1) is a sub-unit for preparing, as the chemical product cp(2), an aqueous solution comprising sulfuric acid, wherein the concentration of the sulfuric acid is C_{H2SO4}(2), wherein the dynamic storage means DS(2) of the unit U(2) is a dynamic storage tank for storing the aqueous solution comprising sulfuric acid which is obtained as a chemical product cp(2) in the sub-unit SU(2).

35. The chemical production assembly of any one of embodiments 17 to 32, further comprising a unit for producing an amino toluene, preferably a diamino toluene, arranged downstream of the unit for nitrating toluene, as defined in any one of embodiments 18, 29, 33 and 34,and further comprising a unit for producing a toluene isocyanate, preferably a toluene diisocyanate, arranged downstream of said unit for producing an amino toluene, preferably a diamino toluene, said chemical production assembly preferably further comprising a unit for producing phosgene, arranged upstream of the unit for producing a toluene isocyanate, preferably a toluene diisocyanate.

36. A process for producing an isocyanate, being carried out in a chemical production assembly according to any one of embodiments 1 to 35.

37. The process of embodiment 36, being a process for producing a diisocyanate, preferably for producing a toluene diisocyanate, more preferably for producing one or more of 2,4-toluene diisocyanate (TDI) and 2,6-toluene diisocyanate.

38. Use of a chemical production assembly according to any one of embodiments 1 to 35 and/or of a process according to embodiment 36 or 37 for increasing the interruption-free operation time of an isocyanate production process, wherein the isocynate is preferably a diisocya-nate, more preferably a toluene diisocyanate, more preferably one or more of 2,4-toluene diisocyanate (TDI) and 2,6-toluene diisocyanate.

In the context of the present invention, the units for PR_{N}(i), PR_{N}(i+1), IR_{N}(i) and IR_{N}(i+1) are given in t/t, meaning metric tons of sulfuric acid per hour/metric tons of nitrated product per hour.

Usually, the unit according to the invention will be comprised in a production plant, preferably in a production plant for producing an isocyanate, more preferably for producing a diisocyanate, more preferably for producing a toluene diisocyanate, more preferably for producing one or more of 2,4-toluene diisocyanate (TDI) and 2,6-toluene diisocyanate. If a mixture comprising 2,4-tolu-ene diisocyanate and 2,6-toluene diisocyanate is produced, the molar ratio of 2,4-toluene diiso-cyanate relative to 2,6-toluene diisocyanate is preferably in the range of from 1:1 to 6:1, more preferably in the range of from 1:1 to 5:1, such as in the range of from 1:1 to 2:1 like 1.5:1, or such as in the range of from 3.5:1 to 4.5:1 like 4:1. Further, it is conceivable that said diisocyanate comprises or is methylene diphenylisocyanate (MDI) and/or hexamethylene diisocyanate (HDI).

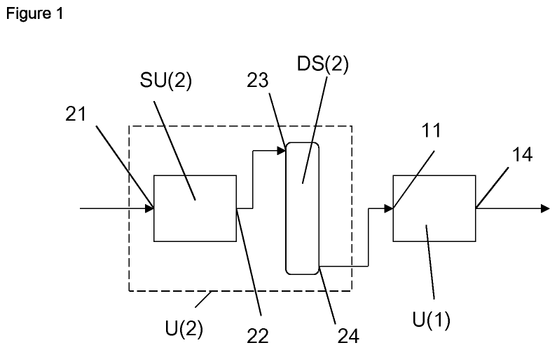

The present invention is further illustrated by the following examples and FIGS. 1 to 4.

FIG. 1, a first example of the invention, shows two units U(1) and U(2) of a chemical production assembly according to a first example of the invention. These two units U(1) and U(2) could be the only units of the chemical production assembly, but often they are parts of a longer production chain. The first unit U(1) comprises an inlet means 11 and an outlet means 14 and the second unit U(2) also comprises an inlet means 21 and an outlet means 24. The second unit U(2) is comprises of at least to elements, namely a sub-unit SU(2) and a dynamic storage means DS(2). The inlet means 21 of the second unit U(2) is identical with the inlet means 21 of the sub-unit SU(2) and the outlet means 24 of the dynamic storage is identical with the outlet means of the second unit U(2). The sub-unit SU(2) and the dynamic storage means DS(2) are connected to one another and thus the sub-unit SU(2) comprises an outlet means 22 and the dynamic storage device DS(2) comprises an inlet means 23.

The sub-unit SU(2) receives a chemical product through its inlet means 21 and prepares a chem-ical product cp(i+1) at a preparation rate PR(i+1). This chemical product cp(i+1) provided to the second unit U(1) through the dynamic storage means DS(2), which does not alter the chemical product cp(i+1). The first unit U(i) receives the product cp(i+1) at an input rate IR(i) and uses it for preparing a chemical product cp(i). Because of the presence of the dynamic storage device DS(2), the preparation rate PR(i+1) and the input rate IR(i) do not need to be identical and thus the unit U(i) can be maintained while the sub-unit SU(2) keeps preparing chemical product cp(i+1) and vice versa.

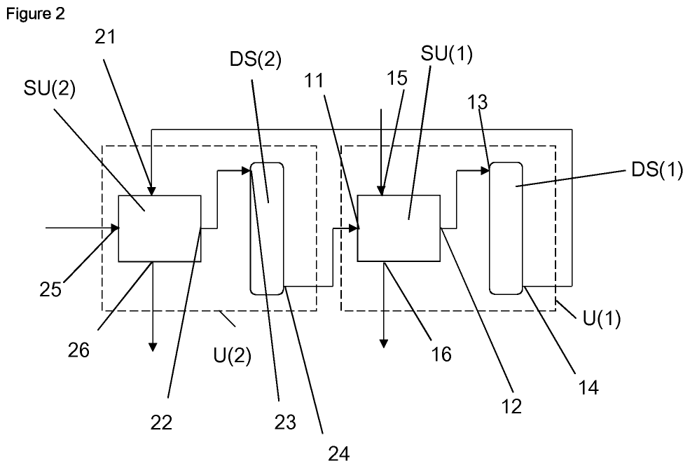

As is shown in the second example of the invention according to FIG. 2, the two units U(1) and U(2) can form a cycle such that unit U(1) is upstream as well as downstream of unit U(2) and both units comprise a sub-unit SU(1), SU(2) and a dynamic storage device DS(1), DS(2). In the em-bodiment shown, both units U(1), U(2) show an additional inlet means 15, 25 and an additional outlet means 16, 26. For example, unit U(2) can serve for cleaning and/or concentrating a liquid catalyst that is used by unit U(1).

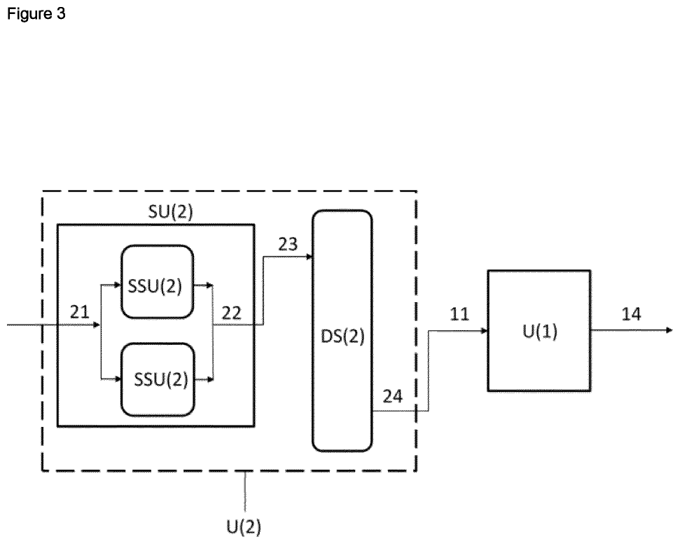

FIG. 3 relates to a third example showing 2 sub-units SSU(2) that are arranged in parallel forming SU(2). The remaining labels of FIG. 3 are according to FIG. 1.

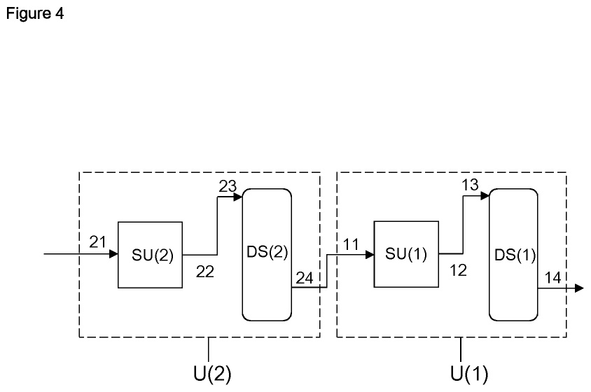

FIG. 4 relates to a fourth example showing a unit U(1), comprising a sub-unit SU(1) and a dy-namic storage means DS (1) and a unit U(2) comprising a sub-unit SU(2) and a dynamic storage means DS(2). The inlet means 11 of the unit U(1) is identical with the inlet means 11 of the sub-unit SU(1) and the inlet means 11 provides chemical product cp(i+1) to unit U(1) and in par-ticular SU(1). The outlet means 12 of sub-unit SU(1) provides the chemical product cp(i) to the dynamic storage means DS(1) through the inlet means 13. The outlet means 14 of the dynamic storage DS(1) is identical with the outlet means of the unit U(1). The remaining labels of FIG. 4 are according to FIG. 1. AMENDMENTS TO THE CLAIMS

1.-16. (Cancelled)

17. A chemical production assembly for producing an isocyanate, comprising n serially arranged units U(i), i=1 . . . n, n>2, wherein (a) a unit U(i) is for preparing a chemical product cp(i) at a preparation rate PR(i) by using, as starting material, a chemical product cp(i+1) prepared in the unit U(i+1) arranged upstream of said unit U(i), wherein said unit U(i) comprises an inlet means for receiving said chemical product cp(i+1) at an input rate IR(i), said unit U(i) being characterized by a nominal preparation rate PRN(i) and a nominal input rate IRN (i);
(b) a unit U(i+1), i=1 . . . n-1, is for preparing the chemical product cp(i+1) and for supplying said chemical product cp(i+1) to the inlet means of the unit U(i) arranged downstream of said unit U(i+1) at a supply rate SR(i+1) with SR(i+1)=IR (i);
(c) at least one unit U(i+1) comprises (c.1) a sub-unit SU(i+1) for preparing the chemical product cp(i+1), wherein said sub-unit SU(i+1) comprises an inlet means for receiving a chemical product at an input rate IR(i+1) and an outlet means for removing said chemical product cp(i+1) from SU(i+1) at a preparation rate PR(i+1), said sub-unit SU(i+1) being characterized by a nominal preparation rate PRN(i+1) with PRN(i+1)/IRN (i) and a nominal input rate IRN(i+1); and
(c.2) a dynamic storage means DS(i+1) for temporary storage of chemical product cp(i+1) prepared according to (c.1), wherein said dynamic storage means DS(i+1) comprises an inlet means being connected to the outlet means of SU(i+1) for receiving chemical product cp(i+1) from SU(i+1), and further comprises an outlet means for removing chemical product cp(i+1) from DS(i+1) at the supply rate SR(i+1), said outlet means being connected to the inlet means of the unit U(i) arranged downstream of U(i+1), said dynamic storage means DS(i+1) having a storage capacity SC(i+1) and being characterized by a dynamic storage rate DR(i+1) with DR(i+1)=PR(i+1)−SR(i+1);
wherein for said at least one unit U(i+1) according to (c), DR(i+1)/0 if at least one of the ratios IR (i): IRN(i) and IR(i+1): IRN(i+1) is in the range of from 0.95:1 to 1.05:1.

18. The chemical production assembly of claim 17, wherein each unit U(i) exhibits a maintenance mode which is characterized by IR(i)=PR(i)=0, and a working mode which is characterized by IR(i)=IRN(i) and PR(i)=PRN(i).

19. The chemical production assembly of claim 18, wherein at least one unit U(i) according to (a) exhibits a regular maintenance pattern with a regular maintenance time Δt_{MM}(i) for which the unit U(i) is in its maintenance mode.

20. The chemical production assembly of claim 17, wherein at least one sub-unit SU(i+1) according to (c.1) exhibits a regular maintenance pattern with a regular maintenance time Δt_{MM}(i+1) for which the sub-unit SU(i+1) is in its maintenance mode.

21. The chemical production assembly of claim 20, wherein the regular maintenance pattern of the at least one unit U(i) according to (a) exhibits a maintenance interval time Δt_{MI}(i) between two consecutive maintenance times Δt_{MM}(i) for which the at least one unit U(i) is not in its maintenance mode, and the regular maintenance pattern of the at least one sub-unit U(i+1) according to (c.1) exhibits a maintenance interval time Δt_{MI}(i+1) between two consecutive maintenance times Δt_{MM}(i+1) for which the at least one sub-unit SU(i+1) is not its in maintenance mode, wherein SC(i+1) >min [Δt_{MI}(i),Δt_{MI}(i+1)] x |DR(i+1) |.

22. The chemical production assembly of claim 17, wherein for at least one unit U (i) according to (a) and at least one unit U(i+1) according to (b), - the at least one unit U(i) exhibits an average input rate IRA(i) and an average preparation rate PRA(i) with IRA(i) <IRN(i) and PRA(i) <PRN (i); and
-the at least one sub-unit SU(i+1) according to (c.1) exhibits an average input rate IRA(i+1) and an average preparation rate PRA(i+1) with IRA(i+1) <IRN(i+1) and PRA(i+1) <PRN(i+1);
and IRA(i)=PRA(i+1).

23. The chemical production assembly of claim 17, being a production assembly for producing a diisocyanate.

24. The chemical production assembly of claim 17, comprising a unit U(i) according to (a) for preparing, as the chemical product cp(i), an aqueous solution comprising sulfuric acid with the concentration of the sulfuric acid being CH2SO4(i), by using, as the starting material cp(i+1), an aqueous solution comprising sulfuric acid with the concentration of the sulfuric acid being CH2SO4 (1+1), wherein CH2SO4(i+1) <CHsSO4(i).

25. The chemical production assembly of claim 24, wherein the sub-unit SU(i+1) of the unit U(i+1) arranged upstream of the unit U(i) is a sub-unit for nitrating an organic compound, with an aqueous solution comprising nitric acid in the presence of sulfuric acid as a catalyst, wherein an aqueous solution comprising sulfuric acid is obtained as a chemical product cp(i+1), wherein in said obtained aqueous solution, the concentration of the sulfuric acid is CH2SO4(i+1), wherein the dynamic storage means DS(i+1) of the unit U(i+1) is a dynamic storage tank for storing the aqueous solution comprising sulfuric acid which is obtained as a chemical product cp(i+1) in the sub-unit SU(i+1).

26. The chemical production assembly of claim 17, comprising a unit U(i) according to (a) for nitrating an organic compound, with an aqueous solution comprising nitric acid in the presence of sulfuric acid as a catalyst, wherein an aqueous solution comprising sulfuric acid is obtained as a chemical product cp(i), wherein in said obtained aqueous solution, the concentration of the sulfuric acid is CH2SO4(i) by using, as the starting material cp(i+1), an aqueous solution comprising sulfuric acid, wherein the concentration of the sulfuric acid is CH2SO4(i+1), with CH2SO4(i+1) >CH2SO4(i).

27. The chemical production assembly of claim 26, wherein the sub-unit SU(i+1) of the unit U(i+1) arranged upstream of the unit U(i) is a sub-unit for preparing, as the chemical product cp(i+1), an aqueous solution comprising sulfuric acid, wherein the concentration of the sulfuric acid is CH2SO4 (1+1), wherein the dynamic storage means DS(i+1) of the unit U(i+1) is a dynamic storage tank for storing the aqueous solution comprising sulfuric acid which is obtained as a chemical product cp(i+1) in the sub-unit SU(i+1).

28. The chemical production assembly of claim 23, comprising a unit U(i+1) and a unit U(i), wherein for at least one unit U(i) according to (a) and at least one unit U(i+1) according to (b), - the at least one unit U(i) exhibits an average input rate IRA(i) and an average preparation rate PRA(i) with IRA(i) <IRN(i) and PRA(i) <PRN (i); and
-the at least one sub-unit SU(i+1) according to (c.1) exhibits an average input rate IRA(i+1) and an average preparation rate PRA(i+1) with IRA(i+1) <IRN(i+1) and PRA(i+1) <PRN(i+1);
and IRA(i)=PRA(i+1), wherein the unit U(i) according to (a) is a unit for nitrating an organic compound, with an aqueous solution comprising nitric acid in the presence of sulfuric acid as a catalyst, wherein an aqueous solution comprising sulfuric acid is obtained as a chemical product cp(i), wherein in said obtained aqueous solution, the concentration of the sulfuric acid is CH2SO4(i) by using, as the starting material cp(i+1), an aqueous solution comprising sulfuric acid, wherein the concentration of the sulfuric acid is CH2SO4 (1+1), with CH2SO4(i+1) >CH2SO4(i).

29. The chemical production assembly of claim 25, further comprising a unit for producing an amino toluene, arranged downstream of the unit for nitrating an organic compound, and further comprising a unit for producing a toluene isocyanate, arranged downstream of said unit for producing an amino toluene, and further comprising a unit for producing phosgene, arranged upstream of the unit for producing a toluene isocyanate.

30. The chemical production assembly of claim 17, with n=2, comprising 2 serially arranged units U(1) and U(2), wherein (a) unit U(1) is for preparing a chemical product cp(1) at a preparation rate PR (1) by using, as starting material, a chemical product cp(2) prepared in the unit U(2) arranged upstream of said unit U (1), wherein said unit U(1) comprises an inlet means for receiving said chemical product cp(2) at an input rate IR (1), said unit U(1) being characterized by a nominal preparation rate PRN (1) and a nominal input rate IRN (1);
(b) unit U(2), is for preparing the chemical product cp(2) and for supplying said chemical product cp(2) to the inlet means of the unit U(1) arranged downstream of said unit U(2) at a supply rate SR(2) with SR(2)=IR (1);
(c) the unit U(2) comprises (c.1) a sub-unit SU(2) for preparing the chemical product cp(2), wherein said sub-unit SU(2) comprises an inlet means for receiving a chemical product at an input rate IR(2) and an outlet means for removing said chemical product cp(2) from SU(2) at a preparation rate PR(2), said sub-unit SU(2) being characterized by a nominal preparation rate PRN(2) with PRN(2) ¥ IRN (1) and a nominal input rate IRN(2); and
(c.2) a dynamic storage means DS(2) for temporary storage of chemical product cp(2) prepared according to (c.1), wherein said dynamic storage means DS(2) comprises an inlet means being connected to the outlet means of SU(2) for receiving chemical product cp(2) from SU(2), and further comprises an outlet means for removing chemical product cp(2) from DS(2) at the supply rate SR(2), said outlet means being connected to the inlet means of the unit U (1) arranged downstream of U(2), said dynamic storage means DS(2) having a storage capacity SC(2) and being characterized by a dynamic storage rate DR(2) with DR(2)=PR(2)-SR(2);
wherein for said unit U(2) according to (c), DR(2) #0 if at least one of the ratios IR (1): IRN (1) and IR(2): IRN(2) is in the range of from 0.95:1 to 1.05:1.

31. A process for producing an isocyanate, being carried out in a chemical production assembly according to claim 17.

32. A method comprising utilizing the chemical production assembly according to claim 17 for increasing the interruption-free operation time of an isocyanate production process.
